(19) United States
(12) Plant Patent Application Publication
(10) Pub. No.: US 2009/0193550 P1
(43) Pub. Date: Jul. 30, 2009

(54) CHESTNUT PLANT NAMED 'AU BUCK I"

(50) ;

(75) Inventors: W. Alfred Dozier, JR., Opelika, AL (US); J. D. Norton, (US); Curtis J. Hansen, Opelika, AL (US)

Correspondence Address: HAVERSTOCK & OWENS LLP, 162 N WOLFE ROAD, SUNNYVALE, CA, 94086, US

(73) Assignee: Auburn University, an Alabama Corporation

(21) Appl. No.: 12/012,091

(22) Filed: Jan. 30, 2008

(51) Int. Cl. A01H 5/00

(52) U.S. Cl. PLT/152

(57) ABSTRACT

‘AU Buck I’ is a new and distinct Chinese chestnut cultivar that is precocious, produces a large crop annually of small to medium sized nuts that mature early and the nuts start dropping about August 28. Nut drop continues for a 4-5 week period. The nuts mature and start dropping 3-4 weeks before most cultivars. The early nut drop and small to medium nut size of ‘AU Buck I’ make it ideal for use as a food source for wildlife. ‘AU Buck I’ is a medium to tall tree with a height of 8.53 meters and a smaller canopy area (62.74 square feet) than most Chinese chestnut trees. ‘AU Buck I’ is the first cultivar to mature and drop nuts in a series of four Chinese chestnut cultivars that will provide for a continuous nut drop of a high energy wildlife food source from late August through mid-to-late November.

### LATIN NAME OF THE GENUS AND SPECIES OF THE PLANT CLAIMED

Castanea P. Mill., Castanea mollissima Blume.

### VARIETY DENOMINATION

‘AU BUCK I’

### BACKGROUND OF THE INVENTION

A Chinese chestnut planting was established at Auburn University, Auburn, Ala., from nuts collected in China. The planting was established on the United States Department of Agriculture Horticulture Farm which in later years became the Mainstation Horticulture Farm. Precocious and prolific-bearing, blight resistant seedlings were selected for nut appearance, size and quality. Each generation of seedlings were the product of controlled mass pollination from the most promising seedlings selected from the previous generation. ‘AU Cropper’, ‘AU Leader’ and ‘AU Homestead’ were released from a second generation of approximately 2000 seedlings. A planting of third generation seedlings from controlled mass pollination of ‘AU Leader’, ‘AU Homestead’ and ‘AU Cropper’ was established at the Auburn University Piedmont Substation at Camp Hill, Ala.

### SUMMARY OF THE INVENTION

‘AU Buck I’ is an open pollinated seedling of ‘AU Cropper’.

The present invention relates to a new and distinct Chinese chestnut cultivar that is blight resistant, precocious, produces a heavy crop annually, produces small to medium sized nuts (9.3 g) and begins nut drop about August 28 and continues for a 4-5 week period. The nuts mature and start dropping 3-4 weeks earlier than most chestnut cultivars. The large crop of small to medium sized nuts is an excellent food source for wildlife such as deer, turkey and squirrel. The 15-year old ‘AU Buck I’ tree is a medium-sized tree that is 8.53 meters tall with a canopy width of 8.94 meters and a canopy area of 62.74 square meters. The ‘AU Buck I’ tree produced nuts the third year after transplanting, has produced a heavy crop annually, and produced 66.5 kg (146.5 lbs) of nuts in 2006. There has been no evidence of insect or disease damage to the tree, foliage, or nuts. ‘AU Buck I’ is the first cultivar to mature and drop nuts in a series of four Chinese chestnut cultivars that will provide a continuous nut drop of a high energy wildlife food source from late August through mid-to-late November. ‘AU Buck II’, ‘AU Buck III’ and ‘AU Buck IV’ are the other three cultivars, which are disclosed in U.S. patent application Ser. No. ______, filed on ______, and entitled “CHESTNUT PLANT NAMED ‘AU BUCK II’” [Attorney Docket No. AUB-07200], U.S. patent application Ser. No. ______, filed on ______, and entitled “CHESTNUT PLANT NAMED ‘AU BUCK III’” [Attorney Docket No. AUB-07300] and U.S. patent application Ser. No. ______, filed on ______, and entitled “CHESTNUT PLANT NAMED ‘AU BUCK IV’” [Attorney Docket No. AUB-07400], which are hereby incorporated by reference.

The new cultivar is able to be asexually reproduced by budding or grafting onto a seedling Chinese chestnut rootstock. The unique characteristics come true to form and are established and transmitted through asexual propagation.

### BRIEF DESCRIPTION OF THE DRAWINGS

FIG. 1 is a photograph of nuts of the ‘AU BUCK I’ cultivar.

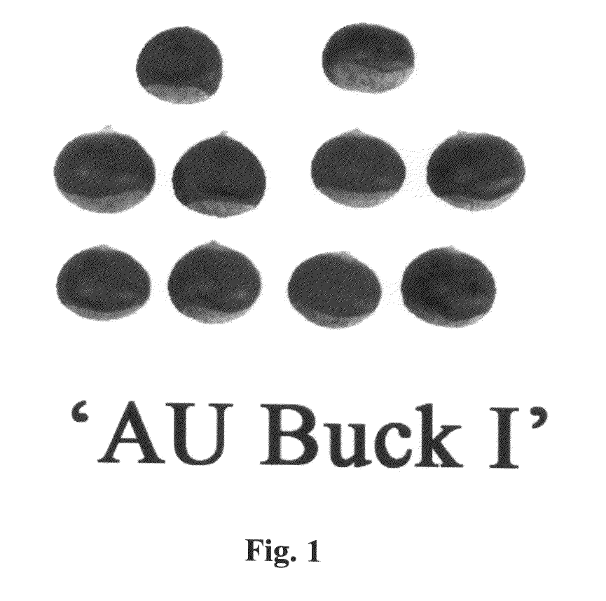

FIG. 2 is a photograph of nuts of the ‘AU BUCK I’ cultivar.

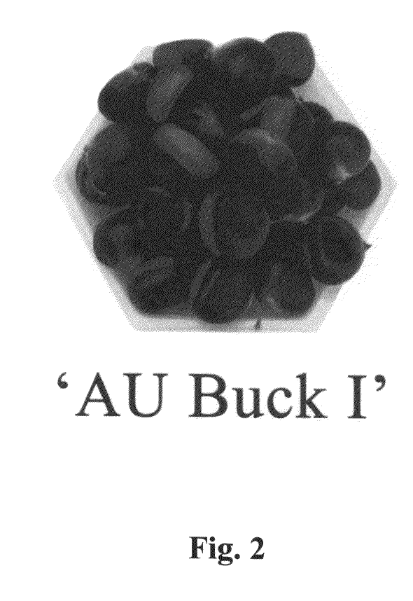

FIG. 3 is a photograph of a tree of the ‘AU BUCK I’ cultivar.

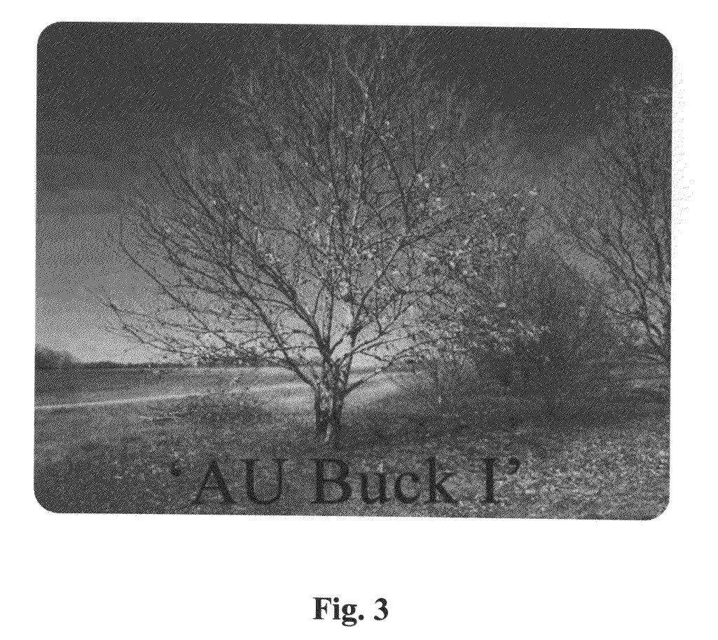

### DETAILED BOTANICAL DESCRIPTION

The Chinese chestnut, Castanea mollissima Blume, is a cold, hardy, temperate zone species native to China. It can be grown between 30° and 50° latitudes. The Chinese chestnut is resistant to chestnut blight fungus Cryphonectria parasitica. Generally, Chinese chestnuts are grown on a wide range of soils, but well-drained, deep and fertile soils are considered the best. Soils should be slightly acidic with pH 5.6-6.5. The name mollissima means soft hair and this species is recognized by dense hair on young leaves and downy yellow terminal parts of the shoots in winter. The leaf blade is thicker and, in general, mature leaves are broader than those of other species. The nuts have a small scar or hilum. The pellicle or thin membranous skin on the nuts is thin and peels readily from the kernel. The trees are a spreading type and long-lived with a round top. The trees have bark with furrows and buds with 3-4 scales and leaves are 2 ranked, serrated with numerous parallel veins.

Chestnuts are monoecious and staminate flowers appear on erect cylindrical catkins with 10-20 stamens and 6-parted calyx. Pistillate flowers are borne on lower part of the upper staminate catkins and rarely on separate catkins, usually 3 in a prickly symmetrical involucre with 7-9 styles and a 6-celled ovary. Nuts are small, brown with a pale scar at the base. Generally, 1-3 nuts per involucre or bur are present.

‘AU Buck I’ is a medium tall tree that has a broadly oval shape and low, dense, and spreading branches. The original 15-year old tree of ‘AU Buck I’ is 8.53 meters (28 feet) tall with a trunk diameter at breast height of 25.4 cm (10.0 inches). The canopy is 8.94 meters (29.31 feet) wide and covers an area of 62.74 square meters (638 square feet). The average nut length is 26.1 mm (1.03 inches) and the average nut width is 30.4 mm (1.20 inches). The nuts start dropping about August 28 and continue to drop for 4-5 weeks. The tree is very prolific, fruits annually, and starts dropping nuts about 3-4 weeks before most cultivars.

In the planting at the Piedmont Substation, Camp Hill, Ala., accurate yields could not be obtained due to extremely heavy wildlife (deer and turkey) feeding. Therefore, the trees were rated for crop load each year. ‘AU Buck I’ has been rated with a large crop load each season. In the fall of 2006, individual trees were caged with 6 foot (1.83 meters) tall chicken wire prior to nut drop to exclude wildlife, and nuts were picked up daily during nut drop. The original 15-year old tree of ‘AU Buck I’ produced 66.5 kg (146.5 pounds) of nuts in 2006. This yield is above the reported yields for similar-aged trees.

The table below illustrates the specific differences between the ‘AU BUCK I’ cultivar and the ‘REVIVAL’ cultivar.

| The botanical details of this new and distinctive variety of | The botanical details of this variety of |
|---|---|
| chestnut tree-with color definitions (except those in | chestnut tree-with color definitions (except those in |
| common color terms) referenced to Royal Horticultural | common color terms) referenced to Maerz and Paul |
| Society's Colour Chart (RHS) and color was also determmined | Dictionary of Color-are as follows: |
| using an electronic spectrophotometer to determine hue angle | |
| and Chroma (spectrophotometer model CM-2002; Minolta Camera Co., Japan). | |
| ‘AU BUCK 1’ CHESTNUT | ‘REVIVAL’ |
| Tree: | Tree: |
| Size (at maturity)-small-fifteen year old original tree is 8.53 meters tall | Size (at maturity)-large |
| with a canopy width of 8.94 meters and a canopy area of 62.74 sq. meters | |
| Tree shape is broadly oval with dense, low and spreading branches. | |
| Vigor-very vigorous | Vigor-very vigorous |
| Trunk: | Trunk: |
| Form-uproght, spreading with branches low and dense | Form-upright with branches spreading in upper |
| | reaches of tree. |
| Texture-relatively smooth | Texture-relatively smooth |
| Color of bark-grey-brown RHS N199A, Chroma C* 15.89, hue angle 91.08 | Color of bark-Silvergray (13-A-1) |
| Branches: | Branches: |
| Form-spreading | Form-strong |
| Texture-relatively smooth | Texture-relatively smooth |
| Lenticels-few, small | Lenticels-few, small |
| Branching habit-dense, low and spreading | Branching habit-spreading in upper region of tree |
| Color-new wood: grey-brown, RHS N199B, Chroma C* 14.72, | Color-new wood: reddish brown and glossy, mature |
| hue angle 79.23, mature wood: grey-brown, RHS N199B | wood: solver gray |
| Chroma C* 14.02, hue angle 93.86 | |
| Foliage: | Foliage: |
| Quantity-abundant | Quantity-abundant |
| Density-dense | Density-dense |
| Leaves: | Leaves: |
| Size-large. Length (cm) 17.1 (14.1-23.1) [20] | Size-large. Average length-5-7″ (including petiole). |
| width (cm) 7.5 (5.8-9.2) [20] | Average width-2″ |
| leaf ratio 2.3 (1.9-2.7) [20] | |
| Shape-elliptic to ovate | Shape-oblong with acute tip and rounded base |
| leaf tip-acute to acuminate | |
| leaf base-rounded; oblique | |
| Thickness-thick. Leaf venation 1° pinnate: 2° ± parallel, prominent abaxially | Thickness-thick |
| Texture-coriaceous (thick) | Texture-smooth |
| Margin-serrate; with small to medium teeth, spreading | Margin-denate |
| Petiole-length 1.0 cm (0.7-1.3) [20] | Petiole-length: medium. Thckness: medium. |
| Petiole pubescence-glabrous to sparsely hairy | |
| Color-adaxial surface, glabrous blade; glabrous veins, rarely with | Color-Top side-glossy dark green (22-L-12). |
| sparse somple hairs, dark green; shiny, RHS 147A, | Under side-lighter green (21-D-7). |
| Chroma C* 12.32, hue angle 122.62 | |
| abaxial surface, moderately hairy, stellate hairs on blade, glabrous veins, | |
| RHS 148B, Chroma C* 19.97, hue angle 109.24 | |
| Bloom: | Bloom: |
| Amount of bloom-heavy | Amount of bloom-heavy |
| Color-at anthesis, 161D greyed-yellow group, 157D green-white group, | Color-cream white (17-B-1) |
| 155C white group | |
| Blooming period-mid-May. After foliation in April. | Blooming period-late. After leaf out in April |
| Age at which tree starts flowering-early, 2-3 years after graft | Age at which tree starts flowering-early; 2-3 years |
| replacement. | years after graft replacement. |
| Male flower-Catkin length (cm)-14.8 (11.4-17.5) [3] | |
| Male flower-stamen number per catkin-11.1 (10-12) [10] | |
| Female flower-flower number per hbur-3 | |
| Female flower-style number per flower 8 (7-9) [3] | |
| Crop: | Crop: |
| Bearing-regular annual bearer | Bearing-regular (yearly) bearer |
| Productivity-prolific | Productivity-prolific |
| Ripening period-long, nuts begin dropping August 28 and continues for a | Ripening period-short. September 15-October 1. |
| 4-5 week period. | |
| Distribution of nuts on tree-well distributed, fruits on terminals with | Distribution of nuts on tree-well distributed |
| 2-4 burs per terminal. | |
| Tenacity-burs crack while on tree and nuts release and drop from bur. | Tenacity-burrs crack while on tree and nuts easily |
| | release, many falling by themselves |
| Hull: | Hull: |
| Description-spiny round bur | Description-spiny, round burr |
| Size-1.99-2.32″ in diameter | Size-3-4″ in diameter |
| Number of nuts-2-3 per bur | Number of nuts-2-3 per burr |
| Dehiscence-splits easily when still on tree | Dehiscence-splits easily when still on tree. Some |
| | entire burrs split and fall to ground |
| Color-yellow-green at dehiscence, RHS N144C | Color-brown (15-A-8) |
| Nut: | Nut: |
| Size-medium; average size-1.03″ × 1.22″ × 0.67″. Average weight-9.3 grams- | Size-large. Average size-1⅛″ × 1⅛″ × 1″ thick. |
| 49 (46-54) nuts per pound. | Average weight-24-32 nuts per pound |
| Form-flattened on 1 side, hemispheric on other side; basal end nearly flat | Form-broad and ovoid on one side, flat on other side |
| to broadly rounded; apex broadly rounded with a small tip. | |
| Blossom end-small pointed tip | Blossom end-pointed tip |
| Basal end-flattened | Basal end-flattened |
| Color-dark brown, lustrous; RHS 200B, Chroma C* 12.34, hue angle 42.90 | Color-India Red (7-L-6). |
| Pubescence-long and short hairs, simple, fina and white restricted to the distal | |
| ⅛ end of the nut, sparse, glabrate to glabrous elsewhere | |
| Shell-thin | Shell-thin |
| Hardness of shell-relatively hard, yet not rigid | Hardness of shell-relatively hard, yet not rigid |
| Texture of shell-smooth | Texture of shell-smooth |
| Percentage of kernel to nut-very high-90-95% | Percentage of kernel to nut-very high (95%) |
| Kernel: | Kernel: |
| Size-almost as large as nut size | Size-almost as large as nut size |
| Form-same as nut shape | Form-same as nut shape |
| Pellicle-thin | Pellicle-thin |
| Flavor-excellent, very sweet | Flavor-excellent, very sweet. |
| Color-light straw color, RHS 162A, Chroma C* 48.81, hue angle 82.35 | Color-Oyster white (10-B-1) |
| Resistance to insects: no unusual susceptibilities noted | Resistance to insects: no unusual susceptibilities noted |
| Resistance to disease: no susceptibilities to disease noted | Resistance to disease: very high inherent resistance to |
| | chestnut bark fungus (Endothia parasitica), no |
| | other susceptibilities to any other diease |
| The chestnut tree and its nuts herein described may | The chestnut tree and its nuts herein described may |
| vary in slight detail to climatic and soil conditions | vary in slight detail due to climatic and soil conditions |
| under which the variety may be grown; the present | under which the variety may be grown; the present |
| description being of the variety as grown in Camp Hill, | description being of the variety as grown in Alachua, |
| Ala. | Fla. |

What is claimed is:

1. A new and distinct cultivar of the species Castanea mollissima Blume named ‘AU BUCK I’ as described and illustrated herein.
